(19) United States
(12) Patent Application Publication
(10) Pub. No.: US 2021/0259395 A1
(43) Pub. Date: Aug. 26, 2021

(54) SUPPORT STRAP WITH INTEGRATED WINDLASS TOURNIQUET

(71) Applicant: Emergency Products and Research, Kent, OH (US)

(72) Inventors: Kasey P. Larson, Audubon, MN (US); Jason A. Thompson, Springfiled, PA (US); Jim Doherty, Sarasota, FL (US)

(21) Appl. No.: 16/795,651

(22) Filed: Feb. 20, 2020

(51) Int. Cl. A45F 3/14; A61B 17/132

(52) U.S. Cl. CPC A45F 3/14; F41C 33/002; A45F 2003/142; A61B 17/1327

(57) ABSTRACT

A support strap including a band, a windlass strap and a windlass handle. The band has a buckle end and a lead end, and the windlass strap is secured to the band at a first positioned and a second position along the length of the wand. The windlass handle provides a windlass bar and the windlass handle is secured to the windless strap at the position of the windlass bar. Rotation of the windlass handle will rotate the windlass bar and wind the windlass strap.

### FIELD OF THE INVENTION

Generally, the invention relates to a support strap that may also be used as a tourniquet. More particularly, the invention relates to a support strap and tourniquet system comprising a windlass handle that provides a windlass. More specifically, the invention relates to a support strap comprising a band and a windlass strap secured to the band, with a windlass interacting with the band such that, when the windlass is rotated, the windlass strap will wind or twist a portion of the windlass strap so as to provide a means of compression for the band.

### BACKGROUND OF THE INVENTION

A tourniquet uses circumferential compression around a limb to compress the blood vessels of the limb against bone. A tourniquet should be placed along the length of the limb between the injury and the heart. Successful use of a tourniquet results in occlusion of blood flow, thereby helping to control bleeding.

Tourniquet designs are rather simple and use some form of mechanical advantage to achieve the required amount of circumferential compression. There are four common mechanical methods, those being pneumatic, windlass, ratchet, and elastic. As healthcare practices continue to evolve, particularly in the prehospital sector, efforts are being made to ensure that people are trained in the use of bleeding control practices and to encourage them to carry tourniquets and bleeding control supplies with them at all times. This is standard practice among professionals in dangerous fields wherein there is a high probability of suffering a potentially life-threatening injury at work and, consequently, it is not unusual to find tourniquets secured to their uniform or gear.

Although there is a need, and many individuals desire, to be prepared to treat traumatic life-threatening bleeding, there is also a desire to avoid being considered a doomsayer who lives life prepared for conflict at all times. Therefore, there is a need in the art for a tourniquet device that can be worn in public without drawing attention to the fact that the device is a tourniquet. Comfort is also of significant importance, as is the ability for the tourniquet device to be incorporated into a variety of carried items and/or to be incorporated into an item of clothing.

### SUMMARY OF THE INVENTION

In a first embodiment, the present invention provides a support strap comprising: a band having a buckle end and a lead end; a windlass strap secured to said band at a first position and a second position along the length of the band; and a windlass handle providing a windlass bar, said windlass handle being secured to said windlass strap at said windlass bar, such that rotation of said windlass handle rotates said windlass bar and winds said windlass strap.

In a second embodiment, the present invention provides a support strap as in any embodiment above, wherein rotation of said windlass handle rotates said windlass bar so as to twist a portion of said windlass strap which shortens an effective length of said windlass strap.

In a third embodiment, the present invention provides a support strap as in any embodiment above, wherein the strap further comprises a cover strap selectively covering said windlass handle and said windlass strap.

In a fourth embodiment, the present invention provides a support strap as in any embodiment above, wherein said cover strap and said windlass strap are a unitary piece of material.

In a fifth embodiment, the present invention provides a support strap as in any embodiment above, wherein said windlass handle has a flat body capable of lying flush with said band.

In a sixth embodiment, the present invention provides a support strap as in any embodiment above, further comprising at least one storage flap foldable over said windlass handle when said windlass handle is flush with said band, said cover strap and said at least one storage flap securable to each other to cover said windlass handle and said windless strap.

In a seventh embodiment, the present invention provides a support strap as in any embodiment above, wherein the cover strap and storage flap are securable to one another with a hook and loop fastener.

In an eighth embodiment, the present invention provides a support strap as in any embodiment above, further comprising at least one windlass handle retainer having a slot for receiving and retaining a portion of said flat body of said windlass handle, said cover strap and said at least one storage flap securable to one another to cover said windlass handle, said windless strap and said at least one windlass handle retainer.

In a ninth embodiment, the present invention provides a support strap as in any embodiment above, wherein said at least one windlass handle retainer is formed of a flat body capable of lying flush with said band.

In a tenth embodiment, the present invention provides a support strap as in any embodiment above, further comprising at least one windlass handle retainer having a slot for receiving and retaining a portion of said windlass handle to prevent counter-rotation thereof under a force generated by said windlass strap when wound by said windlass bar.

In an eleventh embodiment, the present invention provides a support strap as in any embodiment above, wherein said at least one windlass handle retainer includes a second slot for receiving and retaining a portion of said windlass handle to prevent counter-rotation thereof under a force generated by said windlass strap when wound by said windlass bar, said slot, when employed to retain a portion of said windlass handle, preventing counter-rotation of said windlass handle in a first direction, and said second slot, when employed to retain a portion of said windlass handle, preventing counter-rotation of said windlass handle in an opposite direction from said first direction.

In a twelfth embodiment, the present invention provides a support strap as in any embodiment above, wherein the first slot is open in a first direction and the second slot is open in a second direction, and wherein the first direction is opposite of the second direction.

In a thirteenth embodiment, the present invention provides a support strap as in any embodiment above, wherein said at least one windlass handle retainer includes a first windlass handle retainer and a second windlass handle retainer, said windlass bar of said windlass handle being positioned between said first windlass handle retainer and said second windlass handle retainer.

In a fourteenth embodiment, the present invention provides a support strap as in any embodiment above, further comprising a buckle located at the buckle end of the band.

In a fifteenth embodiment, the present invention provides a support strap as in any embodiment above, wherein the buckle is selected from the group consisting of screw closure type, clamp closure type, stitch closure type, tongue buckle type, double side type, reversible type, double ring type, auto-grip buckle type, military buckle type, clip buckle type, wrestling buckle type, auto-lock buckle type, pin buckle type, flip closure buckle type, self-defense buckle type, automatic locking splice buckle type, military utility buckle type, automatic utility buckle type, webbing buckle type, clip-on buckle type, clip-latch buckle type, rigger's belt buckle type, designer military buckle type, or any other fastening device or buckle that could be attached to the support strap.

In a sixteenth embodiment, the present invention provides a support strap as in any embodiment above, wherein the buckle is a webbing type buckle.

In a seventeenth embodiment, the present invention provides a support strap as in any embodiment above, wherein said band has an exterior side and an interior side and wherein said windlass strap and said windlass handle are located on the exterior side of said band.

In an eighteenth embodiment, the present invention provides a support strap as in any embodiment above, further comprising a comfort pad located on the interior side of the band at a position opposite said windlass handle.

In a nineteenth embodiment, the present invention provides a support strap as in any embodiment above, further comprising a lanyard connection loop.

In a twentieth embodiment, the present invention provides a support strap as in any embodiment above, further comprising a stabilizer component attached to the support strap.

In a twenty-first embodiment, the present invention provides a support strap as in any embodiment above, wherein the stabilizer component includes a payload chamber or channel accessible through flaps located at both ends of the stabilizer component.

In a twenty-second embodiment, the present invention provides a support strap as in any embodiment above, further comprising at least one windlass handle retainer having a slot for receiving and retaining a portion of said windlass handle and a second slot for receiving and retaining a portion of said windlass handle.

In a twenty-third embodiment, the present invention provides a method of using the support strap as in any embodiment above comprising the steps of: forming a loop that will be placed around an extremity adjacent an injury to said extremity; feeding the lead end of band through the buckle; cinching the band; rotating the windlass handle which rotates the windlass bar and winds the windlass strap; and retaining a portion of the windlass handle within either the slot or second slot of the at least one windlass handle retainer.

In a twenty-fourth embodiment, the present invention provides a method of using the support strap as in any embodiment above comprising the steps of forming a loop that will be placed around an extremity adjacent a hemorrhagic injury, around the pelvis for managing a suspected pelvic fracture or hemorrhagic injury adjacent the pelvis, or around the torso and shoulder joint for managing a hemorrhagic injury involving the shoulder or upper arm; feeding the lead end of band through the buckle; cinching the band; rotating the windlass handle which rotates the windlass bar and winds the windlass strap; and retaining a portion of the windlass handle within either the slot or second slot of the at least one windlass handle retainer.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 is a perspective view of one embodiment of the support strap of the present invention, show with a cover strap and storage flap closed over the tourniquet system portion;

FIG. 2 is a perspective view of the support strap as shown in FIG. 1 with the cover strap and storage flaps open to see the tourniquet system;

FIG. 3 is a perspective view focused in on an embodiment of a tourniquet system;

FIG. 4 is a plan view of one embodiment of a windlass handle of the tourniquet system;

FIG. 5 is a plan view of one embodiment of a windlass handle retainer of the tourniquet system;

FIG. 6 is a perspective view of one embodiment of the support strap of the present invention showing use of a comfort pad at the location of the tourniquet system;

FIG. 7 is a perspective view of one embodiment of the support strap of the present invention utilizing a stabilizer component;

FIG. 8 is a plan view of one embodiment of the stabilizer component of the present invention;

FIG. 9 is a perspective view of one embodiment of the stabilizer component of the present invention wherein the stabilizer component includes a payload chamber;

FIG. 10 of the present invention shows a plan view of one embodiment of the stabilizer component wherein the payload chamber includes a puller and a pull cord capable of pulling a rope;

FIG. 11 of the present invention shows how an embodiment of the support strap of the present invention can be used as a shoulder bag strap;

FIG. 12 of the present invention shows how an embodiment of the support strap of the present invention can be used as a long gun strap;

FIG. 13 of the present invention shows a windlass strap being tightly wound with a portion of a windlass handle being retained within an open slot provided by a windlass handle retainer;

FIG. 14 is a plan view of a second embodiment of a windlass handle of the tourniquet system of the present invention; and

FIG. 15 is a plan view of a second embodiment of a windlass handle retainer of the tourniquet system of the present invention.

### DETAILED DESCRIPTION OF ILLUSTRATIVE EMBODIMENTS

Disclosed are one or more support straps having an integrated tourniquet system and methods of their use. The support strap can be employed as a strap for virtually any item commonly carried by a strap, such as, without limitation, shoulder bags, purses, luggage, long guns, backpacks, and the like. It can also be employed as a belt by securing a lead end through a buckle.

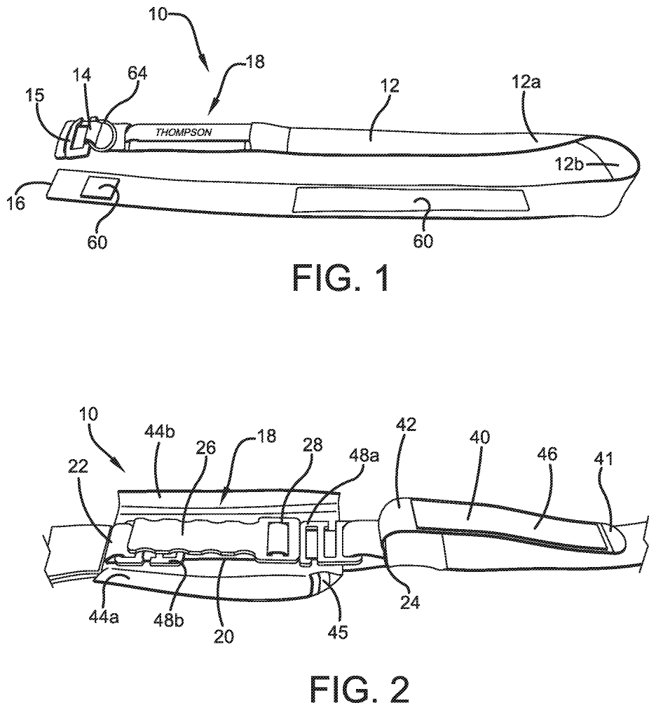

FIG. 1 shows one embodiment of a support strap 10 of the present invention. Support strap 10 comprises a band 12 having a buckle end 14 and a lead end 16. In some embodiments, buckle end 14 and a buckle 15. Band 12 has an exterior side 12a and an interior side 12b. If support strap 10 is used as a daily wear belt, interior side 12b will be located against the waist of a user. As shown in FIGS. 1 and 2, support strap 10 additionally comprises an integrated tourniquet system 18 that can be located anywhere along band 12, but in some embodiments is located proximate buckle end 14 on exterior side 12a of band 12. Locating it proximate buckle end 14 provides easy access and helps the wearer/user easily recall the location. Tourniquet system 18 located proximate buckle end 14 is thus provided at a location where support strap 10 will be manipulated for removal from its use as a support strap or belt, and thus the individual removing it and switching its use to that of a tourniquet will ‘without excessive thought’ have hands at the location of the tourniquet system. Because time is of utmost importance in stopping life-threatening bleeding, this location is thus helpful.

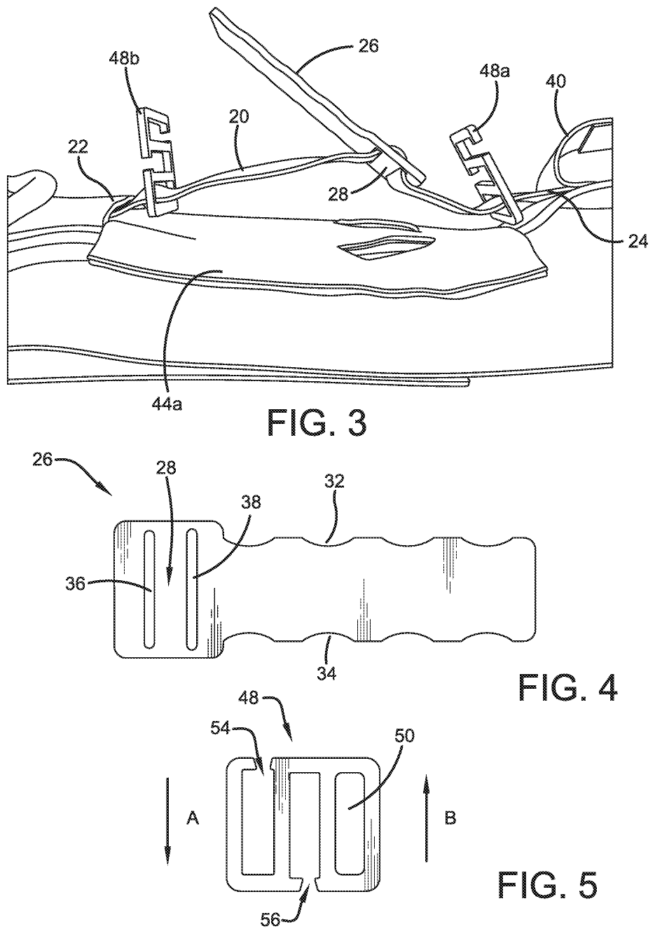

As shown in FIGS. 2 and 3, tourniquet system 18 includes a windlass strap 20 secured to band 12 at a first position 22 and a second position 24 along the length of band 12. Tourniquet system 18 also includes a windlass handle 26 providing a windlass bar 28, with windlass handle 26 being secured to windlass strap 20 at windlass bar 28. As shown in FIG. 4, windlass handle 26 has a flat body 30 such that windlass handle 26 will lie flush against band 12 when stored and not in use. In some embodiments, windlass handle 26 includes a contoured first side 32 opposite a contoured second side 34. Windlass handle 26 includes a first slot 36 positioned adjacent windlass bar 28 and a second slot 38 positioned adjacent windlass bar 28, opposite of first slot 36.

In some embodiments, as shown in FIG. 3, windlass handle 26 is secured to windlass strap 20 by windlass strap 20 extending through first slot 36 from a first side of windlass handle 26, around windlass bar 28 on the other side of windlass handle 26, and then through second slot 38 returning back to the first side. Windlass handle 26 is secured to windlass strap 20 at windlass bar 28 such that rotation of windlass handle 26 rotates windlass bar 28 and winds windlass strap 20 to effectively shorten the distance between first position 22 and second position 24, effectively shortening band 12 to provide a form of circumferential compression when band 12 is secured about a body part and tightened to reduce the circumference of band 12. In some embodiments, prior to rotation of windlass handle 26, band 12 is so secured by having lead end 16 secured through buckle 15 and pulled to secure band 12 tightly about the body part.

In some embodiments, tourniquet system 18 includes a cover strap 40 that selectively covers windlass handle 26 and windlass strap 20. In some embodiments, cover strap 40 and windlass strap 20 originate from one unitary strap 42, such as shown in FIG. 2, with a single tie down 25 (such as stitching) defining the second position 24 and the location where cover strap 40 is secured to band 12. In other embodiments, cover strap 40 and windlass strap 20 can be two separate straps both appropriately secured to band 12.

Tourniquet system 18 furthermore comprises at least one storage flap 44 that folds over windlass handle 26 when windlass handle 26 lies flush with band 12. In some embodiments, tourniquet system 18 includes two storage flaps 44a and 44b, located on opposite sides of band 12, such as shown in FIG. 2. In some embodiments, cover strap 40, when selectively covering windlass handle 26 and windlass strap 20, is secured to the at least one storage flap 44 through the use of a hook-and-loop fastener system. In some embodiments, the at least one storage flap 44 (or 44a, 44b as the case may be) has either hooks or loops (designated in the drawings by numeral 45) on its/their exposed side when folded to cover windlass handle 26, and the cover has either loops or hooks (designated in the drawings by the numeral 46) to appropriately interact therewith. In some embodiments, a single component hook-and-loop fastener, rather than a traditional two-component hook-and-loop fastener, is employed as the hook and loop designated at 45.

The hook-and-loop type fastening is employed in some embodiments to allow for very efficient access to the elements of tourniquet system 18, particularly under the extreme stress of tourniquet application in a life or limb saving situation. Cover strap 40 is simply grabbed and ripped away from the one or more storage flaps 44 and the storage flaps 44 either fall away or are easily brushed aside in accessing windlass handle 26. In embodiments with opposed storage flaps 44a, 44b, they can be made to fold over windlass handle 26 and retainers 48 (disclosed below) without overlapping each other providing their hook and/or loop fastener for interaction with the complementary fastener (designated at 46) on cover strap 40 to fully encase windlass handle 26 and retainers 48.

In some embodiments, tourniquet system 18 includes at least one windlass handle retainer 48 as shown in detail in FIG. 5. The at least one windlass handle retainer 48 is secured to windlass strap 20 between the secured position of windlass handle 26 (at the windlass bar 28) and either first position 22 or second position 24. In some embodiments, the at least one windlass handle retainer 48 is secured by windlass strap 20 extending through a slot 50 located within the at least one windlass handle retainer 48 such that the at least one windlass handle retainer 48 is movable along windlass strap 20. The at least one windlass handle retainer 48 also includes at least one open slot 54. The at least one open slot 54 receives and retains a portion of flat body 30 of windlass handle 26 to prevent counter-rotation of windlass handle 26 under a force generated by windlass strap 20 when windlass strap 20 is wound by windlass bar 28. This relationship between windlass handle 26 and the at least one windlass handle retainer 48 will be explained in further detail below.

In one or more embodiments of the present invention, the at least one windlass handle retainer 48 additionally includes a second open slot 56 that is also capable of receiving and retaining a portion of flat body 30 of windlass handle 26 to prevent counter-rotation of windlass handle 26 under a force generated by windlass strap 20 when windlass strap 20 is wound by windlass bar 28. Open slot 54, when employed to retain a portion of flat body 30 of windlass handle 26, prevents counter-rotation of the windlass handle 26 in a first direction A; and, open slot 56, when employed to retain a portion of flat body 30 of windlass handle 26, prevents counter-rotation of windlass handle 26 in a second direction B, wherein second direction B is in an opposite direction from first direction A. These directions can be thought of as generally clockwise and generally counterclockwise. By having two open slots as shown at open slot 54 and open slot 56, the operator of the tourniquet does not have to be concerned about the direction windlass bar 28 is rotated by windlass handle 26, as the counter-rotation of the windlass can be prevented by choosing the appropriate open slot 54 or 56 for securing of windlass handle 26.

In one or more embodiments of the present invention, system 18 includes two windlass handle retainers 48a and 48b. In said embodiments, windlass bar 28 is positioned on windlass strap 20 between windlass handle retainer 48a and windlass handle retainer 48b. In one or more embodiments of the present invention, to assist in accurately centering windlass bar 28 on windlass strap 20, a centering marking can be placed on windlass strap 20. In such embodiments, the centering marking aids in rotation of windlass bar 28. In such embodiments, the compression provided upon rotation of windlass bar 28 by rotation of windlass handle 26 can be more accurately controlled as windlass handle 26 can be secured upon a half rotation as opposed to another full rotation when additional compression is needed. When there is only one retainer 48, compression may be too little upon reaching the location of a slot 54, 56, but too great upon attempting to reach the location of the slot 54, 56 after another full rotation. Thus, two windlass handle retainers 48a and 48b, each with two slots 54, 56, provide beneficial utility.

In some embodiments, cover strap 40 and at least one storage flap 44 selectively cover and uncover each windlass handle retainer 48 as well as windlass handle 26 and windlass strap 20 so as to effectively hide the fact that a tourniquet system 18 is part of support strap 10.

In some embodiments, the support strap 10 includes a buckle 15 located at the buckle end 14. In some embodiments, band 12 also includes a hook and loop fastener system 60 which is used in combination with buckle 15 so as cinch support strap 10 when in use. Buckle 15 can be virtually any type of belt buckle. In some embodiments, buckle 15 is selected from screw closure buckles, clamp closure buckles, stitch closure buckles, tongue buckles, double side buckles, reversible buckles, double ring buckles, auto-grip buckles, military buckles, clip buckles, wrestling buckle, auto-lock buckles, pin buckles, flip closure buckles, self-defense buckles, automatic locking splice buckles, military utility buckles, automatic utility buckles, webbing buckles, clip-on buckles, clip-latch buckles, rigger's belt buckles, and designer military buckles. If the type of belt buckle selected for buckle 15 requires a piece of equipment on lead end 16 of band 12 or an aperture along the band 12 to be able to cinch, the present invention envisions that such equipment will be located on the lead end 16 or such apertures will be located along the band 12 as required and known in the art. In some embodiments, buckle 15 is a webbing buckle, as seen in FIG. 1, wherein the lead end 16 of the band 12 is capable of being put through buckle 15 at a first slot from the interior side and then brought back over a bar and through a second slot and cinched tight, utilizing hook and loop fastener system 60 to further secure band 12.

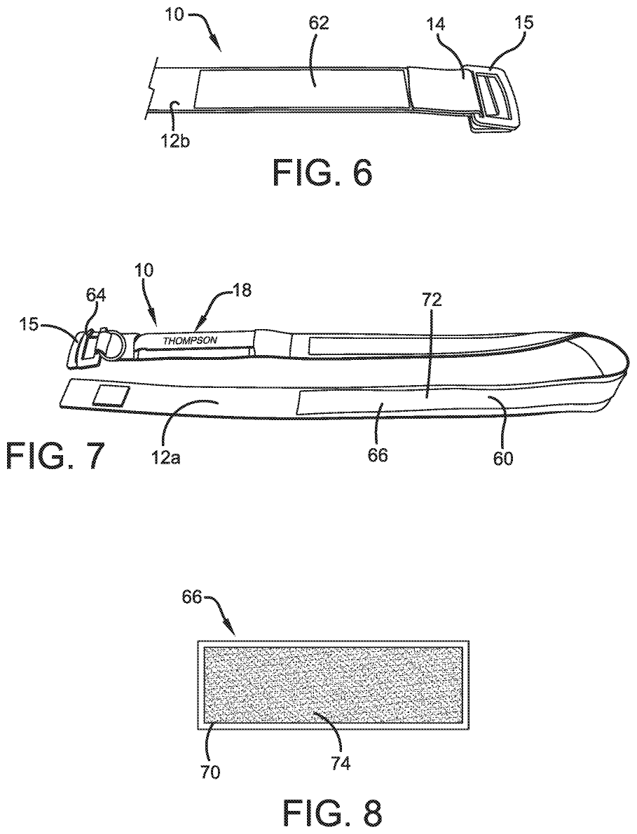

In some embodiments, support strap 10 includes a comfort pad 62 as shown in FIG. 6, secured to interior side 12b of band 12 at a similar position of tourniquet system 18 on exterior side 12a. Comfort pad 62 provides a soft padded portion on interior side 12b of band 12 so as to reduce discomfort that might be experienced by the storage of windlass handle 26 and retainer 48 members.

In some embodiments, support strap 10 includes a connection loop 64. As shown in FIG. 1, connection loop 64 is located proximate buckle 15. Although connection loop 64 is shown proximate buckle 15 of support strap 10, it is also contemplated that connection loop 64 can be located at any position along band 12. Connection loop 64 allows the user of support strap 10 to connect a carabiner lanyard to support strap 10 to protect the user from falling from a platform, out of a helicopter, out of the back of a plane, etc., and connection loop 64 also gives the user the ability to connect a rope to support strap 10 and conduct emergency bailouts when necessary.

In some embodiments, the support strap 10 includes a stabilizer component 66. As shown in FIGS. 7 and 8, stabilizer component 66 is capable of being selectively secured to support strap 10 at a portion of hook-and-loop fastener system 60. Stabilizer component 66 includes an interior side 70 and an exterior side 72. The interior side 70 of the stabilizer component 66 contains a hook-and-loop fastener portion 74 to allow stabilizer component 66 to attach to support strap 10 at a portion of hook and loop fastener system 60. A user utilizing support strap 10 would exploit stabilizer component 66 to allow for heavier items, such as a holster for securing tools, a gun or a knife to support strap 10 when used, for example, as a daily wear belt. Stabilizer component 66 effectively increases the thickness and thus sturdiness of band 12 making the mounting of additional items easier and allowing for heavier items.

In addition, stabilizer component 66 offers additional improvisational capabilities, such as providing edge protection when employing ropes over surfaces that could jeopardize the integrity of a rope, employment as a lift-assist device for providing ergonomic advantages, a human-carrying device that could be used by two or more people to carry a third person, a device that could be looped over a horizontal pole or branch to provide a foundation for hanging fluids for intravenous fluid administration, as well as numerous other possibilities.

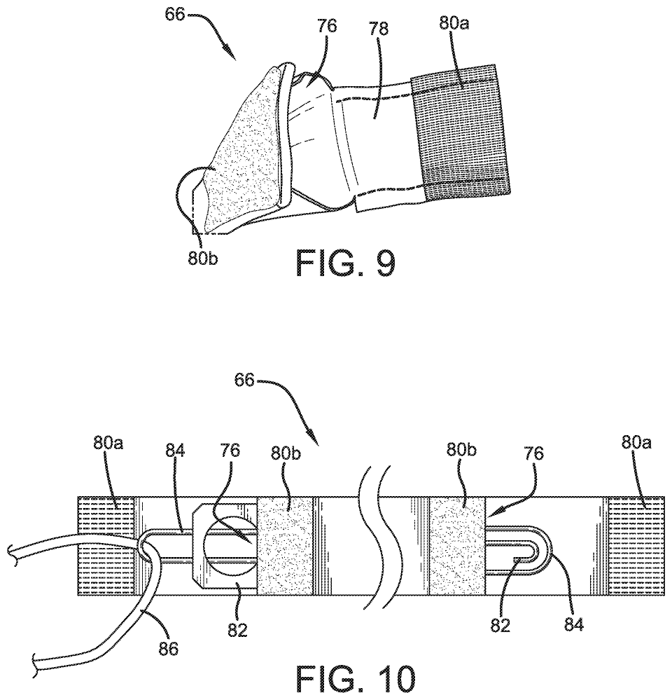

In some embodiments, such as shown in FIG. 9, stabilizer component 66 can include a payload chamber 76 capable of being used to discretely store small items, such as travel documents, cash or first aid supplies, in addition to providing similar capabilities as described above for a stabilizer component 66 that does not contain a payload chamber. In these embodiments, payload chamber 76 is accessible through flaps 78 located at both ends of stabilizer component 66 wherein each flap is equipped with a hook-and-loop fastener portion 80a and the exterior side 72 is equipped with a hook-and-loop fastener portion 80b.

In some embodiments, payload chamber 76 holds an individual first aid kit (IFAK) or individual survival aid kit (ISAK). In some embodiments the IFAK kit includes one or more of hemostatic gauze, chest seals, decompression needles, and/or disposable medical gloves. In some embodiments the ISAK kit includes one or more of survival blanket, water purification tablets, fire starting equipment, fishing line, signaling mirror, and/or spear tip. However, the present invention is not limited by the contents of payload chamber 68. In one or more embodiments, the contents of payload chamber 76 will be vacuum sealed down to the smallest size possible. In some embodiments, when support strap 10 is being used as a daily wear belt, payload chamber 76, filled with its various contents/IFAK/ISAK, extends outward from the surface of band 12 no farther than about 14 mm so that support strap 10 can easily fit through the belt loops of the user's pants. In other embodiments, this distance is no farther than 13 mm, and in other embodiments, no farther than 12 mm.

In some embodiments, such as shown in FIG. 10, payload chamber 76 accommodates a puller 82 that pulls a pull cord 84 that is capable of pulling a rope, such as rope 86. In some embodiments, a rope may be too large to be able to be folded to enable pull cord 84 to pull said rope. In those instances, a large rope can either be pushed through payload chamber 76 or by using a method with pull cord 84 being fashioned to grip the rope and pull it through payload chamber 76.

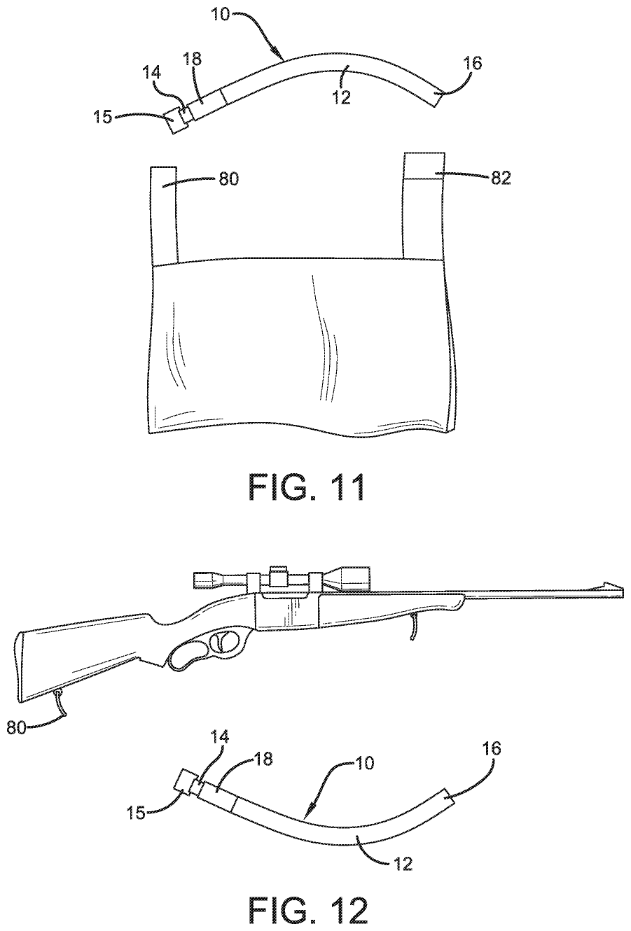

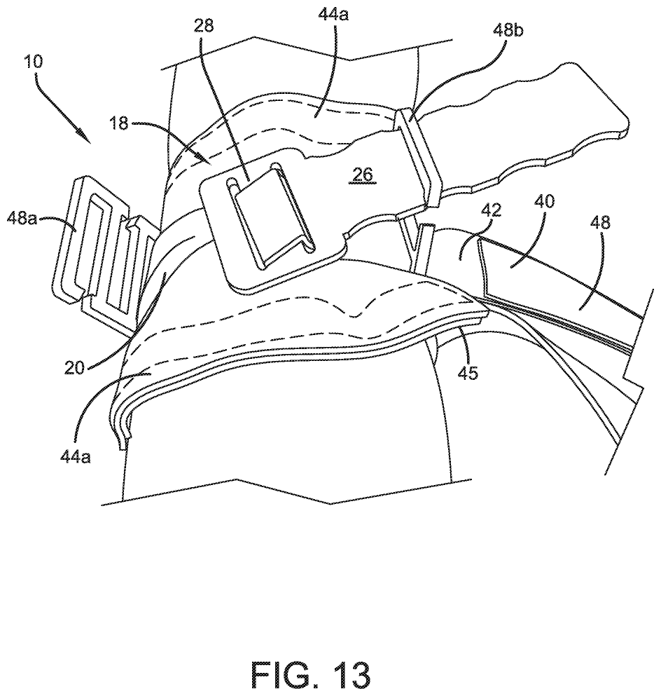

FIGS. 11 and 12 show the versatility of embodiments of support strap 10 and lend visual support to the concept of employing it not only as a belt but also as a support or shoulder strap for other items. In FIGS. 11 and 12 it is contemplated, for purposes of example only, that the support strap 10 can be used as a shoulder bag strap (FIG. 11) and as a long gun strap (FIG. 12). The support strap 10 can be utilized in virtually any situation. If used as a daily wear belt, the buckle end 14 and a lead end 16 will mate with each other appropriately based on the type of buckle chosen. If used as either a shoulder bag strap or a long gun strap, for example, the buckle end 14 will mate with a connector or strap 80 provided on the shoulder bag/long gun, while the lead end 16 will mate with an appropriate connector 82 provided on the shoulder bag/long gun.

The band 12 of the present invention is made of virtually any material that could provide the strength and durability needed for the employment of the support strap 10 as a support strap or belt and as a tourniquet. In some embodiments, the band 12 is made from a material selected from the group consisting of woven nylon (e.g., webbing), leather, plastic, rubber, cotton, polyester webbing with a thermoplastic polyurethane (TPU) or polyvinyl chloride (PVC) coating and/or another suitable material that can withstand tension forces sufficient to cut off blood flow. In some embodiments, the band 12 is made of woven nylon.

In some embodiments, band 12 is defined as having a breaking strength of at least about 500 pounds; in other embodiments at least about 3,500 pounds; and in yet other embodiments at least about 3,750 pounds. In some embodiments, band 12 is defined as having a breaking strength of at most about 5,000 pounds; in other embodiments at most about 4,500 pounds; and in yet other embodiments at most about 4,250 pounds. In some embodiments, band 12 is defined as having a breaking strength of between about 3,000 and about 5,000 pounds; in other embodiments between about 3,500 and about 4,500 pounds; and in yet other embodiment between about 3,750 and about 4,250 pounds. In some embodiments, band 12 has a breaking strength of about 4,000 pounds.

In some embodiments, band 12 has a length, wherein the length is the distance between the buckle end 14 and the lead end 16, of at least about 800 mm; in other embodiments of at least about 900 mm; and in yet other embodiments at least about 1000 mm. In some embodiments, band 12 has a length of at most about 1400 mm; in other embodiment at most about 1300 mm; and in yet other embodiments at most about 1200 mm. In some embodiments, band 12 has a length of between about 800 mm and about 1400 mm; in other embodiments between about 900 mm and about 1300 mm; and in yet other embodiments between about 1000 mm and about 1200 mm. In some embodiments, band 12 has a length of about 1100 mm.

In some embodiments, band 12 has a width of at least about 30 mm; in other embodiments at least about 34 mm; and in yet other embodiments at least about 36 mm. In some embodiments, band 12 has a width of at most about 60 mm; in other embodiments at most about 48 mm; and in yet other embodiments at most about 45 mm. In some embodiments band 12 has a width of between about 30 mm and about 60 mm; in other embodiments between about 34 mm and about 48 mm; and in yet other embodiments between about 36 mm and about 45 mm. In some embodiments, band 12 has a width of about 38 mm.

Windlass strap 20 of the present invention is of virtually any material that could provide the strength and durability needed for the employment of the windlass strap 20 as the cinching element of a tourniquet. In some embodiments, windlass strap 20 is made from a material selected from the group consisting of woven nylon (e.g., webbing), leather, plastic, rubber, cotton, polyester webbing with a thermoplastic polyurethane (TPU) or polyvinyl chloride (PVC) coating and/or another suitable material that can withstand tension forces sufficient to cut off blood flow. In some embodiments, windlass strap 20 is made of woven nylon.

In some embodiments, windlass strap 20 is defined as having a breaking strength of at least about 500 pounds; in other embodiments at least about 3,500 pounds; and in yet other embodiments at least about 3,750 pounds. In some embodiments, windlass strap 20 is defined as having a breaking strength of at most about 5,000 pounds; in other embodiments at most about 4,500 pounds; and in yet other embodiments at most about 4,350 pounds. In some embodiments, windlass strap 20 is defined as having a breaking strength of between about 3,000 and about 5,000 pounds; in other embodiments between about 3,500 and about 4,500 pounds; and in yet other embodiment between about 3,750 and about 4,000 pounds. In one embodiment, windlass strap 20 has a breaking strength of about 4,000 pounds.

In some embodiments, windlass strap 20 has a length, wherein the length is the distance between first position 22 and second position 24, of at least about 100 mm; in other embodiments of at least about 120 mm; and in yet other embodiments at least about 140 mm. In some embodiments, windlass strap 20 has a length of at most about 200 mm; in other embodiment at most about 180 mm; and in yet other embodiments at most about 160 mm. In some embodiments, windlass strap 20 has a length of between about 100 mm and about 200 mm; in other embodiments between about 120 mm and about 180 mm; and in yet other embodiments between about 140 mm and about 160 mm. In one embodiment, windlass strap 20 has a length of about 150 mm.

In some embodiments, windlass strap 20 has a width of at least about 15 mm; in other embodiments at least about 19 mm; and in yet other embodiments at least about 23 mm. In some embodiments, windlass strap 20 has a width of at most about 35 mm; in other embodiments at most about 31 mm; and in yet other embodiments at most about 27 mm. In some embodiments windlass strap 20 has a width of between about 15 mm and about 35 mm; in other embodiments between about 19 mm and about 31 mm; and in yet other embodiments between about 23 mm and about 27 mm. In one embodiment, windlass strap 20 has a width of about 25 mm.

In some embodiments, windlass strap 20 is located at a first position 22 and a second position 24 wherein the first position 22 and the second position 24 can be positioned at virtually any position across band 12. In some embodiment, windlass strap 20 is located proximate buckle end 14 on exterior side 12a of band 12.

The windlass strap 20 can be secured at first position 22 and second position 24 by virtually any suitable strong and durable securement to withstand the force exerted on those secured positions during use as a tourniquet. In some embodiments, windlass strap 20 is secured at first position 22 and second position 24 through fastening selected from the group consisting of rivets, staples, stitching, or any combination thereof. In some embodiments, windlass strap 20 is secured at first position 22 and second position 24 through the use of stitching. In some embodiments, the type of stitching employed that meets applicable ANSI, OSHA, and CSA requirements for fall protection equipment.

Windlass handle 26 of the present invention is made of virtually any material that could provide the structure needed for use within tourniquet system 18 of support strap. In one or more embodiments, windlass handle 26 is made from a material selected from the group consisting of plastics, metals, composites, and/or other substantially frictionless materials. In some embodiments, windlass handle 26 is made from a metal. In one or more embodiments, windlass handle 26 is made of stainless steel.

In some embodiments, windlass handle 26 has a length that is smaller than the length of windlass strap 20, which, as mentioned above, has a length defined as the distance between first position 22 and second position 24. In some embodiments, windlass handle 26 has a length of at least about 70 mm; in other embodiments of at least about 80 mm; and in yet other embodiments at least about 90 mm. In some embodiments, windlass handle 26 has a length of at most about 130 mm; in other embodiments at most about 120 mm; and in yet other embodiments at most about 110 mm. In some embodiments, windlass handle 26 has a length of between about 70 mm and about 130 mm; in other embodiments between about 80 mm and about 120 mm; and in yet other embodiments between about 90 mm and about 110 mm. In some embodiments, windlass handle 26 has a length of about 100 mm.

In some embodiments, windlass handle 26 has a width that is smaller than the width of band 12. In this way, windlass handle 26 is readily and comfortably held concealed with a cover strap 40 and remains streamlined with the remainder of support strap 10. In some embodiments, windlass handle 26 has a width of at least about 13 mm; in other embodiments at least about 17 mm; in other embodiments at least about 21 mm; in other embodiments at least about 25 mm; and in yet other embodiments at least about 28 mm. In some embodiments, windlass handle 26 has a width of at most about 33 mm; in other embodiments at most about 29 mm; and in yet other embodiments at most about 25 mm. In some embodiments windlass handle 26 has a width of between about 13 mm and about 33 mm; in other embodiments between about 17 mm and about 29 mm; and in yet other embodiments between about 21 mm and about 25 mm. In one embodiment, windlass handle 26 has a width of about 23 mm. Windlass handle 26 need not be contoured as shown in the figures, but, in some instances, it is contoured to provide a good grip for one employing it as a tourniquet.

Cover strap 40 of the present invention is made of virtually any material that could provide the structure needed for use as a protective cover. In some embodiments, cover strap 40 is made from a material selected from the group consisting of woven nylon (e.g., webbing), leather, plastic, rubber, cotton, polyester webbing with a thermoplastic polyurethane (TPU) or polyvinyl chloride (PVC) coating and/or another suitable material that can withstand tension forces sufficient to cut off blood flow. In one embodiment, cover strap 40 is made of woven nylon.

In some embodiments, cover strap 40 has a length and width suitable, in interaction with the at least one storage flap 44, to cover the elements of tourniquet system 18, including one or more of or all of windlass handle 26, the at least one windlass handle retainer 48, and windlass strap 20.

In some embodiments, the cover strap 40 is secured at second position 24 through the use of a securing means selected from the group consisting of rivets, staples, stitching, or any combination thereof. In one embodiment, cover strap 40 is secured at second position 24 through the use of stitching.

In one or more embodiments, the at least one storage flap 44 is secured along one of the two side edges of band 12 through the use of a securing means selected from the group consisting of rivets, staples, stitching, or any combination thereof. In one embodiment, storage flap 44 is secured through the use of stitching.

In some embodiments, two storage flaps 44a and 44b, located on opposite sides of band 12, such as shown in FIG. 2a, are utilized to mate with cover strap 40 so as to selectively cover windlass handle 26 and windlass strap 20. In such embodiments, storage flaps 44a and 44b are made of virtually any material suitable for the role of assisting in covering and protecting the tourniquet system components retained.

The at least one windlass handle retainer 48 of the present invention is made of virtually any material that could provide the strength and durability needed for its use in securing the windlass handle against counter-rotation when the support strap 10 is used as a tourniquet. In one or more embodiments, the at least one windlass handle retainer 48 is made from a material selected from the group consisting of plastics, metals, composites, and/or other substantially frictionless materials. In one embodiment, the at least one windlass handle retainer 48 is made from a metal. In one or more embodiments, the at least one windlass handle retainer 48 is made from stainless steel.

In some embodiments, the at least one windlass handle retainer 48 has length and width dimensions suitable so that the at least one windlass handle retainer 48 can fit within the containment formed by the cover strap 40 and the at least one storage flap 44. This will generally entail being of less width than the band 12.

Comfort pad 62 of the present invention is made of virtually any material that could provide the structure needed for use within support strap 10. In one or more embodiments, comfort pad 62 is made from a material selected from the group consisting of foam, rubber, elastomer, cotton, wool, and/or another suitable material that can provide comfort to the use of support strap 10. In some embodiments, comfort pad 62 is made of foam or rubber. In some embodiments, comfort pad 62 has a length and width suitable to provide protection against the pressure of the components of tourniquet system 18 and is therefore sized at the same or slightly larger length or width. In some embodiments, comfort pad 62 is the same size as or slightly smaller in width than the band 12.

In some embodiments, stabilizer component 66 of the present invention is made of virtually any material that could provide the added structural stability previously disclosed with respect to this element. In some embodiments, stabilizer component 66 is made from a material selected from the group consisting of woven nylon (e.g., webbing), leather, plastic, rubber, cotton, polyester webbing with a thermoplastic polyurethane (TPU) or polyvinyl chloride (PVC) coating and/or another suitable material that can withstand downward forces sufficient to cause slumping of support strap 10. In some embodiments, stabilizer component 66 is made of woven nylon. In some embodiments, stabilizer component 66 stabilizer is made of the same material as band 12.

In some embodiments, stabilizer component 66 is defined as having a breaking strength of at least about 500 pounds; in other embodiments at least about 3,500 pounds; and in yet other embodiments at least about 3,750 pounds. In some embodiments, stabilizer component 66 is defined as having a breaking strength of at most about 5,000 pounds; in other embodiments at most about 4,500 pounds; and in yet other embodiments at most about 4,250 pounds. In some embodiments, stabilizer component 66 is defined as having a breaking strength of between about 3,000 and about 5,000 pounds; in other embodiments between about 3,500 and about 4,500 pounds; and in yet other embodiment between about 3,750 and about 4,250 pounds. In some embodiments, stabilizer component 66 has a breaking strength of about 4,000 pounds.

In some embodiments, stabilizer component 66 has a length of at least about 200 mm; in other embodiments of at least about 300 mm; and in yet other embodiments at least about 400 mm. In some embodiments, stabilizer component 66 has a length of at most about 1,000 mm; in other embodiments at most about 900 mm; and in yet other embodiments at most about 800 mm. In some embodiments, stabilizer component 66 has a length of between about 200 mm and about 1,000 mm; in other embodiments between about 300 mm and about 900 mm; and in yet other embodiments between about 400 mm and about 800 mm.

In some embodiments, stabilizer component 66 has a width of at least about 30 mm; in other embodiments at least about 34 mm; and in yet other embodiments at least about 36 mm. In some embodiments, stabilizer component 66 has a width of at most about 48 mm; in other embodiments at most about 45 mm; and in yet other embodiments at most about 40 mm. In some embodiments stabilizer component 66 has a width of between about 30 mm and about 48 mm; in other embodiments between about 34 mm and about 45 mm; and in yet other embodiments between about 36 mm and about 40 mm. In one embodiment, stabilizer component 66 has a width of about 38 mm.

To wear support strap 10 as a daily use belt around the waist of a user, support strap 10 is laced through the belt loops of the user's pants from the user's left side, around the user's back and to user's right side (or vice versa per individual preference). In some embodiments, when support strap 10 is in position around the waist of the user, the tourniquet system 18 resides just left (or right) of buckle 15 and close to the user's iliac crest on the user's left side/right side without passing entirely through the first belt loop of the user's pants. Keeping tourniquet system 18 partially under the first belt loop of the user's pants, without passing entirely through it, allows support strap 10 to be removed swiftly form the user's belt loops when the use of tourniquet system 18 is needed.

When the use of tourniquet system 18 is needed, support strap 10 needs to be removed from the user's belt loops. Once support strap 10 has been removed and is completely free of the belt loops, support strap 10 can be swiftly transitioned from use as a belt, to use as a tourniquet. First, a loop is formed with support strap 10 around the injured body part, such as an upper or lower extremity. Next, lead end 16 is fed completely through buckle 15, and band 12 is to be cinched tightly around the injured limb. Next, cover strap 40 is unsecured from the at least one storage flap 44 so as to uncover windlass handle 26 and windlass strap 20. With band 12 now cinched as tightly as possible, windlass handle 26 is rotated, in either a clockwise or counterclockwise direction, so as to rotate windlass bar 28 in such direction and thereby twist a portion of windlass strap 20. Windlass bar 28 is to be rotated in either a clockwise or counterclockwise direction until the bleeding has been controlled through achieving occlusion of the blood vessels. With windlass strap 20 tightly wound, a portion of flat body 30 of windlass handle 26 is then to be retained within the at least one open slot 54 provided by windlass handle retainer 48a or 48b, such as shown in FIG. 13.

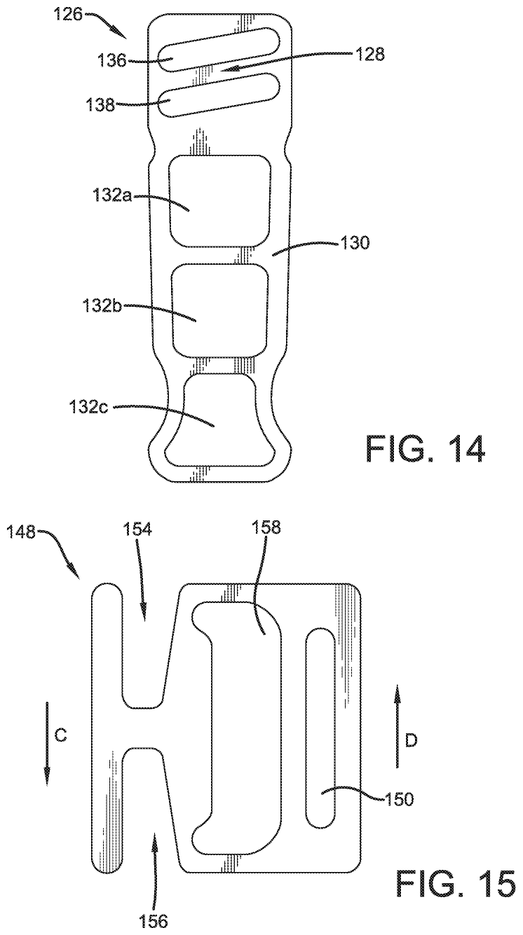

FIG. 14 shows an additional embodiment of a windlass handle of the present invention. FIG. 14 shows a windlass handle 126 providing a windlass bar 128, with windlass handle 126 being securable to a windlass strap 20 at windlass bar 128. As shown in FIG. 14, windlass handle 126 has a flat body 130 such that windlass handle 126 will lie flush against a band 12 when stored and not in use. In some embodiments, windlass handle 126 includes multiple apertures 132a 132b, and 132c located in flat body 130. The multiple apertures 132a, 132b, and 132c lower the overall weight of windlass handle 126 which adds to the comfortability of wearing a support strap 10 including windlass handle 126. Windlass handle 126 includes a first slot 136 positioned adjacent windlass bar 128 and a second slot 138 positioned adjacent windlass bar 128, opposite of first slot 36. As shown in FIG. 14, first slot 136 and second slot 138 run in a diagonal direction through flat body 130, but it is also contemplated that first slot 136 and second slot 138 could run in a straight direction through flat body 130, similar to first slot 26 and second slot 38 in windlass handle 26. The diagonal direction may be found to result in increase applied pressure upon rotation in a direction toward the low end of the slots 136, 138.

In some embodiments, tourniquet system 18 includes at least one windlass handle retainer 148 as shown in detail in FIG. 15. The at least one windlass handle retainer 148 is securable to a windlass strap 20 between a secured position of a windlass handle 26 or 126 (at the windlass bar 28 or 128) and either first position 22 or second position 24. In some embodiments, the at least one windlass handle retainer 148 is secured by windlass strap 20 extending through a slot 150 located within the at least one windlass handle retainer 148 such that the at least one windlass handle retainer 148 is movable along windlass strap 20. The at least one windlass handle retainer 148 also includes at least one open slot 154. The at least one open slot 154 receives and retains a portion of flat body 30 or 130 of windlass handle 26 or 126 to prevent counter-rotation of windlass handle 26 or 126 under a force generated by windlass strap 20 when windlass strap 20 is wound by windlass bar 28 or 128.

In one or more embodiments of the present invention, windlass handle retainer 148 additionally includes an aperture 158 that if present, can serve many purposes such as: (1) receiving and retaining the support strap's lead end, following securing of the windlass handle in a windlass handle retainer 148, to prevent accidental release of the compression achieved through the windlass action; (2) receiving an improvised windlass handle to be employed for achieving mechanical advantage during tourniquet application; and (3) reducing the overall weight of the windlass handle retainer 148. To increase mechanical advantage per (2) above, any suitable strong and long lever arm item that fits could be inserted through the aperture 158 to then engage one of slots 154, 156 and allow the user to apply additional pressure through the lever arm. This could be applied after cinching using the windlass handle 126.

For example, if the tourniquet system 18 of the present invention is utilizing windlass handle retainer(s) 148 and the tourniquet system is needed, support strap 10 needs to be removed from the user's belt loops. Once support strap 10 has been removed and is completely free of the belt loops, support strap 10 can be swiftly transitioned from use as a belt, to use as a tourniquet. First, a loop is formed with support strap 10 around the injured body part, such as an upper or lower extremity. Next, lead end 16 is fed completely through buckle 15, and band 12 is to be cinched tightly around the injured limb. Next, cover strap 40 is unsecured from the at least one storage flap 44 so as to uncover windlass handle 26 or 126 and windlass strap 20. With band 12 now cinched as tightly as possible, windlass handle 26 or 126 is rotated, in either a clockwise or counterclockwise direction, so as to rotate windlass bar 28 or 128 in such direction and thereby twist a portion of windlass strap 20. Windlass bar 28 or 128 is to be rotated in either a clockwise or counterclockwise direction until the bleeding has been controlled through achieving occlusion of the blood vessels. With windlass strap 20 tightly wound, a portion of flat body 30 or 130 of windlass handle 26 or 126 is then to be retained within the at least one open slot 154 provided by windlass handle retainer 148. Finally, as extra assurance for holding the tourniquet system in place, lead end 16 can be fed through aperture 158 and retained therein.

In one or more embodiments of the present invention, the at least one windlass handle retainer 148 additionally includes a second open slot 156 that is also capable of receiving and retaining a portion of flat body 30 or 130 of windlass handle 26 or 126 to prevent counter-rotation of windlass handle 26 or 126 under a force generated by windlass strap 20 when windlass strap 20 is wound by windlass bar 28 or 128. Open slot 154, when employed to retain a portion of flat body 30 or 130 of windlass handle 26 or 126, prevents counter-rotation of the windlass handle 26 or 126 in a first direction C; and, open slot 156, when employed to retain a portion of flat body 30 or 130 of windlass handle 26 or 126, prevents counter-rotation of windlass handle 26 or 126 in a second direction D wherein second direction D is in an opposite direction from first direction C. These directions can be thought of as generally clockwise and generally counter-clockwise. By having two open slots as shown at open slot 154 and open slot 156, the operator of the tourniquet does not have to be concerned about the direction windlass bar 28 or 128 is rotated by windlass handle 26 or 126, as the counter-rotation of the windlass can be prevented by choosing the appropriate open slot 154 or 156 for securing of windlass handle 26 or 126.

In light of the foregoing, it should be appreciated that the present invention significantly advances the art by providing a support strap with an integrated windlass tourniquet that is structurally and functionally improved in a number of ways. While particular embodiments of the invention have been disclosed in detail herein, it should be appreciated that the invention is not limited thereto or thereby inasmuch as variations on the invention herein will be readily appreciated by those skilled in the art. The scope of the invention shall be appreciated from the claims that follow.

What is claimed is:

1. A support strap comprising:
a. a band having a buckle end and a lead end;
b. a windlass strap secured to said band at a first position and a second position along a length of the band; and
c. a windlass handle providing a windlass bar, said windlass handle being secured to said windlass strap at said windlass bar, such that rotation of said windlass handle rotates said windlass bar and winds said windlass strap.

2. The support strap as in claim 1, wherein rotation of said windlass handle rotates said windlass bar so as to twist a portion of said windlass strap which shortens an effective length of said windlass strap.

3. The support strap as in claim 1, further comprising a cover strap selectively covering said windlass handle and said windlass strap.

4. The support strap as in claim 3, wherein said cover strap and said windlass strap are a unitary piece of material.

5. The support strap as in claim 3, wherein said windlass handle has a flat body capable of lying flush with said band.

6. The support strap as in claim 5, further comprising at least one storage flap foldable over said windlass handle when said windlass handle is flush with said band, said cover strap and said at least one storage flap securable to each other to cover said windlass handle and said windlass strap.

7. The support strap as in claim 6, wherein the cover strap and storage flap are securable to one another with a hook and loop fastener.

8. The support strap as in claim 6, further comprising at least one windlass handle retainer having a slot for receiving and retaining a portion of said flat body of said windlass handle, said cover strap and said at least one storage flap securable to one another to cover said windlass handle, said windlass strap and said at least one windlass handle retainer.

9. The support strap as in claim 8, wherein said at least one windlass handle retainer is formed of a flat body capable of lying flush with said band.

10. The support strap as in claim 1, further comprising at least one windlass handle retainer having a slot for receiving and retaining a portion of said windlass handle to prevent counter-rotation thereof under a force generated by said windlass strap when wound by said windlass bar.

11. The support strap as in claim 10, wherein said at least one windlass handle retainer includes a second slot for receiving and retaining a portion of said windlass handle to prevent counter-rotation thereof under a force generated by said windlass strap when wound by said windlass bar, said slot, when employed to retain a portion of said windlass handle, preventing counter-rotation of said windlass handle in a first direction, and said second slot, when employed to retain a portion of said windlass handle, preventing counter-rotation of said windlass handle in an opposite direction from said first direction.

12. The support strap as in claim 11, wherein the first slot is open in a first direction and the second slot is open in a second direction, and wherein the first direction is opposite of the second direction.

13. The support strap as in claim 8, wherein said at least one windlass handle retainer includes a first windlass handle retainer and a second windlass handle retainer, said windlass bar of said windlass handle being positioned between said first windlass handle retainer and said second windlass handle retainer.

14. The support strap as in claim 1, further comprising a buckle located at the buckle end of the band.

15. The support strap as in claim 14, wherein the buckle is selected from the group consisting of screw closure type, clamp closure type, stitch closure type, tongue buckle type, double side type, reversible type, double ring type, auto-grip buckle type, military buckle type, clip buckle type, wrestling buckle type, auto-lock buckle type, pin buckle type, flip closure buckle type, self-defense buckle type, automatic locking splice buckle type, military utility buckle type, automatic utility buckle type, webbing buckle type, clip-on buckle type, clip-latch buckle type, rigger's belt type, and designer military buckle type.

16. The support strap as in claim 15, wherein the buckle is a webbing type buckle.

17. The support strap as in claim 1, wherein said band has an exterior side and an interior side and wherein said windlass strap and said windlass handle are located on the exterior side of said band.

18. The support strap as in claim 17, further comprising a comfort pad located on the interior side of the band at a position opposite said windlass handle.

19. The support strap as in claim 1, further comprising a lanyard connection loop.

20. The support strap as in claim 1, further comprising a stabilizer component attached to the support strap.

21. The support strap as in claim 20, wherein the stabilizer component includes a payload chamber accessible through flaps located at both ends of the stabilizer component.

22. The support strap as in claim 1, further comprising at least one windlass handle retainer having a slot for receiving and retaining a portion of said windlass handle and a second slot for receiving and retaining a portion of said windlass handle.

23. A method of using the support strap of claim 22 as a tourniquet comprising the steps of:
a. forming a loop with the band placed around an extremity adjacent an injury to said extremity;
b. feeding the lead end of band through the buckle;
c. cinching the band;
d. rotating the windlass handle which rotates the windlass bar and winds the windlass strap; and
e. retaining a portion of the windlass handle within either the slot or second slot of the at least one windlass handle retainer.
